(19) United States
(12) Patent Application Publication
(10) Pub. No.: US 2026/0315656 A1
(43) Pub. Date: Oct. 15, 2026

(54) Dynamic Crosslinking System For Cells And Annealed Microgels

(71) Applicant: The Regents of The University of Michigan, Ann Arbor, MI (US)

(72) Inventors: Wahidur RAHMAN, Ann Arbor, MI (US); Sasha Cai LESHER-PEREZ, Ann Arbor, MI (US)

(73) Assignee: The Regents of The University of Michigan, Ann Arbor, MI (US)

(21) Appl. No.: 19/644,678

(22) Filed: Apr. 10, 2026

Related U.S. Application Data
(60) Provisional application No. 63/786,871, filed on Apr. 10, 2025.

(51) Int. Cl. A61L 27/36; A61L 27/18; A61L 27/20; A61L 27/52

(52) U.S. Cl. CPC A61L 27/3687; A61L 27/18; A61L 27/20; A61L 27/52

(57) ABSTRACT

The disclosure provides a dynamic cell support system comprising at least one modified cell (e.g., saccharide or glycoprotein modified) having a modified surface comprising a first functional group. A microgel comprising a second functional group capable of reacting with the first functional group is also part of the cell support system. Thus, the microgel can incorporate the at least one modified cell into the microgel and create the dynamic cell support system. Methods of making dynamic cell support systems are also provided.

### CROSS-REFERENCE TO RELATED APPLICATIONS

This application claims the benefit of U.S. Provisional Application No. 63/786,871, filed Apr. 10, 2025. The entire disclosure of the above application is incorporated herein by reference.

### FIELD

The present disclosure relates to a dynamic cell support system that includes a dynamic crosslinking system for cells and annealed microgels.

### BACKGROUND

This section provides background information related to the present disclosure which is not necessarily prior art.

Microgels, and their composite structures of granular hydrogels, offer modularity, porosity, and injectability, making them ideal for engineered tissue scaffolds. Annealable hydrogels/microporous annealed particle (MAP) gels offer significant advantages in tissue engineering due to their tunable properties While microgels offer significant advantages in tissue engineering, their assembly traditionally relies on static crosslinking methods that lack adaptability and prevent cellular control over microstructure formation. Existing approaches, such as enzyme-mediated crosslinking and UV-initiated thiol-ene chemistry, can introduce cytotoxicity and fail to support dynamic tissue remodeling. Further, rapid and stable microgel assembly is limited by fluid interference (e.g., wound exudates) and a lack of long-term dynamic remodeling. It would be desirable to have a system where cells actively direct microgel assembly for responsive and reconfigurable tissue formation.

### SUMMARY

This section provides a general summary of the disclosure, and is not a comprehensive disclosure of its full scope or all of its features.

In certain aspects, the present disclosure relates to a dynamic cell support system including: at least one modified cell having a modified surface including a first functional group. The dynamic cell support system also includes a microgel including a second functional group capable of reacting with the first functional group to incorporate the at least one modified cell into the microgel and create the dynamic cell support system.

In some aspects, the at least one modified cell has been modified by uptake of a saccharide including the first functional group, wherein the at least one modified cell incorporates the saccharide including the first functional group into its cell membrane.

In some aspects, the first functional group includes an azide and the modified surface is an azide-modified surface.

In some aspects, the first functional group includes tetrazine.

In some aspects, the first functional group is an azide and the second functional group is an alkyne.

In some aspects, the second functional group includes dibenzocyclooctyne or norbornene.

In some aspects, the microgel includes a polymer selected from the group consisting of: hyaluronic acid, polyalkylene glycol, alginate, gelatin, trehalose, and combinations thereof, the polymer further has a functional group or moiety selected from the group consisting of: norbornene, maleimide, cyclononane, bicyclononyne, dibenzocyclooctyne (DBCO), dibenzoazacyclooctyne (DIBAC), vinyl sulfone, azide, tetrazine, acrylate, methacrylate, and combinations thereof.

In some aspects, the microgel includes a polymer selected from the group consisting of: polyalkylene glycol, hyaluronic acid, and combinations thereof.

In some aspects, the microgel includes a polymeric precursor selected from the group consisting of: hyaluronic acid-norbornene, polyethylene glycol-norbornene, polyethylene glycol-bicyclononyne, polyethylene glycol-tetrabicyclononyne, polyethylene glycol-maleimide, polyethylene glycol-di-acrylate, polyethylene glycol-tetra-acrylate, gelatin-norbornene, gelatin-methacrylate, alginate, trehalose, and combinations thereof.

In certain other aspects, the present disclosure relates to a dynamic cell support system including a porous network including a microgel and at least one glycoengineered surface-modified cell attached to the microgel by a chemical bond. The chemical bond is created between a first functional group on a surface of the glycoengineered surface-modified cell reacted with a second functional group on the microgel.

In some aspects, the first functional group includes an azide and the second functional group is an alkyne.

In some aspects, the first functional group includes an azide and the second functional group includes dibenzocyclooctyne (DBCO).

In some aspects, the at least one glycoengineered surface-modified cell is a plurality glycoengineered surface-modified cells and at least a portion of the plurality glycoengineered surface-modified cells serve as cross-linkers between polymer chains in the microgel.

In some aspects, the first functional group is an azide and the second functional group is an alkyne.

In some aspects, the second functional group includes dibenzocyclooctyne or norbornene.

In some aspects, the microgel includes a polymer selected from the group consisting of: hyaluronic acid, polyalkylene glycol, alginate, gelatin, trehalose, and combinations thereof, the polymer further has a functional group or moiety selected from the group consisting of: norbornene, maleimide, cyclononane, bicyclononyne, dibenzocyclooctyne (DBCO), dibenzoazacyclooctyne (DIBAC), vinyl sulfone, azide, tetrazine, acrylate, methacrylate, and combinations thereof.

In some aspects, the microgel includes a polymeric precursor selected from the group consisting of: hyaluronic acid-norbornene, polyethylene glycol-norbornene, polyethylene glycol-bicyclononyne, polyethylene glycol-tetrabicyclononyne, polyethylene glycol-maleimide, polyethylene glycol-di-acrylate, polyethylene glycol-tetra-acrylate, gelatin-norbornene, gelatin-methacrylate, alginate, trehalose, and combinations thereof.

In certain further aspects, the present disclosure relates to a method for making a dynamic cell support system. The method optionally includes reacting a first functional group on at least one glycoengineered surface-modified cell with a second functional group on a microgel or a precursor of the microgel to form a chemical bond between the glycoengineered surface-modified cell and the microgel to create the dynamic cell support system.

In some aspects, the reacting further includes exposing the at least one glycoengineered surface-modified cell and the microgel or precursor of the microgel to an external source of energy or heat.

In some aspects, the microgel or precursor of microgel includes a plurality of microparticles and the at least one glycoengineered surface-modified cell includes a plurality of at least one glycoengineered surface-modified cells and the reacting forms a cellularized microgel network.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1B a variation for forming a dynamic cell support system according to certain aspects of the present disclosure. FIG. 1A shows modifying a microgel by reacting a norbornene group by reacting with a tetrazine on a DBCO (3-arm)-polyethylene glycol-tetrazine molecule. FIG. 1B shows glycoengineering of cells by updates of a modified saccharide so that azide (N₃) groups are presented on a surface. FIG. 1B also shows reaction of the DBCO-modified microgel microparticles with the azides on the glycoengineered cells by a “cellular annealing” process to form a dynamic cell support system including a cellularized microgel.

FIG. 2 shows modification of a microgel precursor by reacting a microgel with various functional groups according to certain aspects of the present disclosure.

FIG. 3 shows a mechanism of glycoengineering cells with an azide-containing glycoprotein on a cell surface and how it can be verified using a DBCO-probe according to certain aspects of the present disclosure.

FIGS. 4A-4C show a buoyancy-driven droplet generator for creating microgel precursor droplets to produce microgel networks for use in accordance with certain aspects of the present disclosure. FIG. 4A shows a schematic of a portion of microfluidic system for microdroplet generation in accordance with certain aspects of the present disclosure. FIG. 4B shows a detailed view of an outlet region of a microfluidic channel of the microfluidic system of FIG. 4A. FIG. 4C shows an outlet region of a microfluidic channel having multiple branches forming multiple microfluidic channel outlets.

FIGS. 5A-5D show fluorescent labeling of microgels with Azide-Fluor488. FIGS. 5A-5B show unmodified microgels with no green fluorescence (FIG. 5A), with the corresponding merged transmitted light and Azide-Fluor488 channel shown in FIG. 5B. FIGS. 5C-5D show DBCO-modified microgel beads stained with AzideFluor 488 exhibiting strong green fluorescence due to azide-DBCO click labeling as visualized in a single-plane confocal microscopy scan (FIG. 5C) and FIG. 5D shows this merged with corresponding transmitted light imaging of the microgel. Scale bars are 300 micrometers.

FIG. 6 shows a confocal microscopy image of a modified cell membrane prepared according to certain aspects of the present disclosure. A first confocal microscopy image shows cells after the first day of feeding with a glycoprotein and a second confocal microscopy image shows cells after a third day of feeding with the glycoprotein. Scale bars are 200 micrometers.

FIG. 7 shows fluorescence intensity of cells with surface azide density is feeding-dependent, enabling tunable control of cell-microgel coupling efficiency. Cells were fed with a glycoprotein Ac4ManNAz) containing solution for 1 to 3 Days (reflecting different feeding regimes).

FIG. 8 shows a phase contrast image of coupled DBCO-modified microgels and azide sugar-fed glycoengineered cells using strain-promoted azide-alkyne cycloaddition (SPAAC) click chemistry.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s) as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

In various aspects, the present disclosure contemplates a dynamic cell support system. The support system may include at least one modified cell having a modified surface comprising a first functional group. The cells may be a single cell type or may include multiple cell types, including prokayrotic cells and/or eukaryotic cells. In certain variations, the cell may be a eukaryotic cell, such as an animal cell. By way of non-limiting example, the cells may be bacterial, animal (including mammalian), plant, fungi, yeast, and the like. The cells may in certain variations include tissue, which may include a collection of cells having a common structure or function. By way of non-limiting example, the cells may be cells for various organs of a human or other animal, such as endothelial cells, cardiac cells, pulmonary cells, neural cells, connective tissue cells, vascular cells, stem cells, immune cells, cancer cells, and the like. Further, the support system includes a microgel comprising a second functional group capable of reacting with the first functional group.

As used herein, the microgel may also be considered to a hydrogel. Microgels may be formed of small particles (e.g., colloids) having at least one dimension on a microscale. Microgels are typically formed of three-dimensional networks of cross-linked polymer chains in a semi-solid or solid phase, which may be microparticles, such as round particles or beads, which may be further reacted with one another in a gelation (or crosslinking or annealing) reaction. In this manner, when the modified cell encounters a region of the microgel, the first functional group can react with the second functional group, for example, in a click chemistry reaction, to form a covalent bond therebetween and thus serving to incorporate the at least one modified cell into the microgel and create the dynamic cell support system.

As will be described further herein, the at least one modified cell may be modified by glycoengineering, by uptake by the cell of a saccharide comprising the first functional group. The saccharide or a portion of the saccharide may then be incorporated on a surface of the cell, such that the first functional group is presented on the surface and the cell is considered to be a glycoengineered surface-modified cell. The first functional group is selected for compatibility and ability to covalently bond with the second functional group, for example, in a click-chemistry reaction, like a strain-promoted azide-alkyne cycloaddition (SPAAC), strain-promoted azide-nitrone cycloaddition (SPANC), Diels-Alder, by way of non-limiting example. In certain variations, the functional group may be an azide. In one variation, the modified saccharide used for glycoengineering may be N-azidoacetylmannosamine-tetraacylated (Ac4ManNAz), which is an azide-containing glycoprotein (a polypeptide having at least one carbohydrate group attached to the polypeptide chain, where the polypeptide thus defines/provides the functional group). After uptake of the saccharide comprising the first functional group, the cell thus incorporates the saccharide comprising the first functional group into its cell membrane where it is presented on the modified surface. In certain variations, the first functional group incorporated into the modified cell comprises an azide (N₃), such that the modified surface of the cell is an azide-modified surface. In certain variations, the first functional group comprises tetrazine.

In certain aspects, the microgel may be formed from a polymer/polymeric backbone that is selected from the group consisting of: hyaluronic acid, polyalkylene glycol (or polyalkylene oxide), such as polyethylene glycol (or polyethylene oxide (PEO)), alginate, gelatin, trehalose, and combinations thereof. In certain aspects, the polymeric backbone is selected from the group consisting of hyaluronic acid, polyalkylene glycol (or polyalkylene oxide), such as polyethylene glycol (or polyethylene oxide (PEO)), and combinations thereof. Suitable polyethylene glycol precursors include 4-arms or 8-arms, by way of non-limiting example.

The polymeric backbone may further have one or more functional groups, for example, including the second functional group. In certain aspects, the polymeric precursor backbone may be functionalized and thus further have a cross-linking or functional group selected from the group consisting of: norbornene, maleimide, cyclononane, bicyclononyne, dibenzocyclooctyne (DBCO), dibenzoazacyclooctyne (DIBAC), vinyl sulfone, azide, tetrazine, acrylate, methacrylate, and combinations thereof. In certain aspects, the functional or reactive group is thus capable of reacting with the crosslinking agent during the gelation reaction. In certain aspects, at least one functional or reactive group is capable of reacting with a crosslinking agent during a crosslinking or annealing reaction that forms the microgel. However, the second functional group incorporated into the polymeric backbone of the microgel is selected to be capable of reacting with the first functional group on the modified cell. In certain aspects, a stoichiometric excess of the second functional group is included on the microgel precursor, so that sufficient amounts of the second functional group remain after the crosslinking or annealing reaction that forms the microgel. In other aspects, the polymeric backbone may comprise multiple distinct functional groups, including the second functional group that reacts with the first functional group on the cell. The second functional group of the microgel may be an alkyne. In certain variations, the second functional group may comprise dibenzocyclooctyne (DBCO) or norbornene. In certain other aspects, the polymeric precursor may be capable of self-crosslinking, for example, alginate can ionically cross-link. In other variations, the polymeric precursor may be trehalose and may dehydrate, for example, for DNA storage or freezing for cell preservation.

In certain variations, the microgel comprises a polymer including a second functional group, which may optionally be functionalized with a functional group for cross-linking, selected from the group consisting of: hyaluronic acid-norbornene, polyethylene glycol-norbornene, polyethylene glycol-bicyclononyne, polyethylene glycol-tetrabicyclononyne, polyethylene glycol-maleimide, polyethylene glycol-di-acrylate, polyethylene glycol-tetra-acrylate, gelatin-norbornene, gelatin-methacrylate, alginate, trehalose, and combinations thereof.

Table 1 sets forth various reactive groups (the pairs of Reactive Group A and Reactive Group B) that can be used for cross-linking in the hydrogel systems contemplated by certain aspects of the present disclosure. It is noted that any polymer backbone described herein can be modified with these reactive groups to make hydrogels.

TABLE 1

| | | | Catalysis/ |
|---|---|---|---|
| | | | Other |
| | | | Materials |
| Chemistry | Reactive Group A | Reactive Group B | Required |
| Ionic crosslinking | Alginate | Calcium | |
| Free radical (UV | Gelatin- | | Photoinitiator, |
| photopolymerization) | methacrylate | | UV |
| | PEG-diacrylate | | |
| Staudinger Ligation | Azide | Triarylphisphine | |
| Cu(I) Catalyzed Azide- | Azide | Alkyne | Copper |
| Alkyne Cycloaddition | | | |
| (CuAAC) | | | |
| Strain-Promoted Azide- | Azide | Cyclooctynes and | |
| Alkyne Cycloaddition | | derivatives | |
| (SPAAC) | | [difluorinated | |
| | | cyclooctyne | |
| | | (DIFO), | |
| | | dibenzocyclooctyne (DBCO)] | |
| | | bicyclononynes | |
| Diels-Alder (DA) Reaction | Diene | dienophile | |
| Inverse-Electron Demand | Tetrazine | Cyclooctene (e.g., | |
| Diels-Alder (IEDDA) | | Norbornene) | |
| Reaction | | | |
| Tandem Cycloaddition- | Azide | trifluoromethyl- | |
| Retro-Diels-Alder (crDA) | | substituted | |
| Reaction | | oxanorbornadiene | |
| Tetrazole-Alkene Photo- | Tetrazole | alkene | |
| Click Reaction | | | |
| Hüisgen 1,3-dipolar | Nitrile oxide or | strained alkene, e.g., | |
| cycloaddition | benzyl nitrile oxide | norbornene | |
| Sulfur (VI) Fluoride | Sulfonyl fluorides | amine or silyl ethers | |
| Exchange (SuFEx) Reaction | or fluorosulfates | | |

Therefore, a precursor material that forms the microgel may comprise a crosslinking agent/crosslinker. In certain variations, the precursor material may comprise a crosslinking agent/crosslinker comprising a thiol group. In other variations, the crosslinking agent may be degradable, for example, by enzymes or hydrolytic degradation. The enzymatically degradable crosslinking agents may further be an MMP (protease)-sensitive peptide or a plasmin-sensitive crosslinker peptide. Various suitable degradable crosslinking agents/crosslinkers, include, by way of example, a crosslinker selected from the group consisting of a dithiothreitol crosslinking agent, a polyethylene-glycol dithiol crosslinking agent, an enzymatically degradable plasmin sensitive peptides with GCYKNSGCYKNSCG (YKNS) crosslinking agent, a hydrolytically degradable ethylene glycol bis(mercaptoacetate) (EGBMA) crosslinking agent, and combinations thereof. In some further aspects, the crosslinking agent is degradable and is one or more an enzymatically degradable plasmin-sensitive YKNS crosslinking agent or a hydrolytically degradable EGBMA crosslinking agent. Suitable glycol dithiol may have an average molecular weight ranging from 1,800 to 3,500. The precursor may also include a catalyst or initiator for initiating the crosslinking or gelation reaction. One suitable example of a photoinitiator is lithium phenyl-2,4,6-trimethylbenzoylphosphinate (LAP).

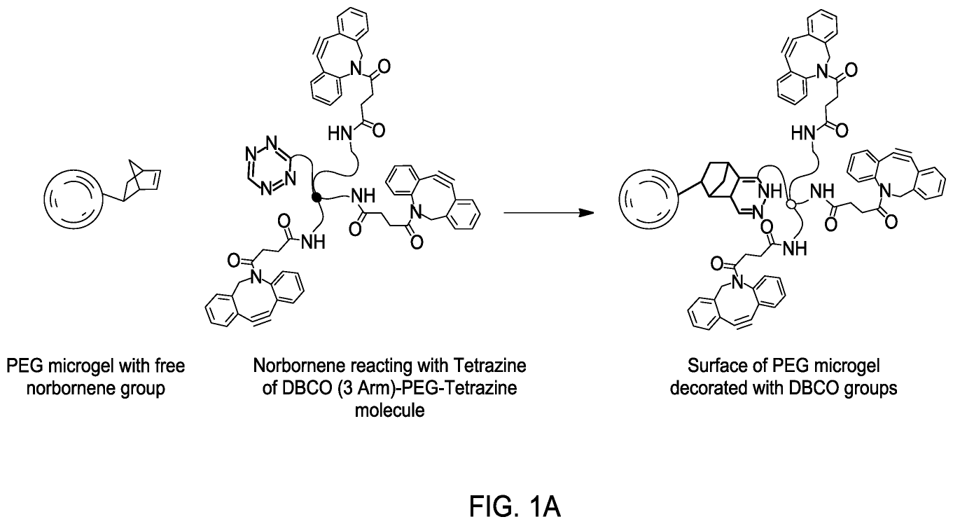

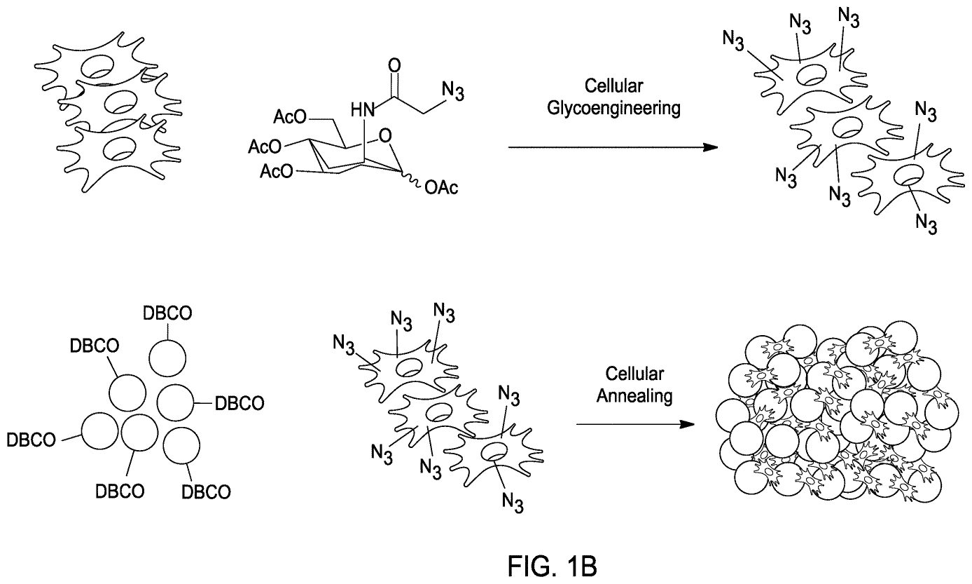

FIGS. 1A-1B show one variation for forming a dynamic cell support system according to certain aspects of the present disclosure. As shown, a microgel precursor in the form of a microparticle (e.g., a microbead, microsphere, or microdroplet) is shown bearing a functional group namely, a norbornene group in FIG. 1A. The PEG microparticle having the norbornene is then reacted with a tetrazine. More specifically, as shown, the tetrazine is part of a dibenzocyclooctyne (DBCO) (3-Arm)-PEG-tetrazine molecule. The norbornene thus reacts with the tetrazine, such that the PEG microgel has a surface decorated with DBCO groups that serve as a second functional group-namely an alkyne. As shown in FIG. 1B, a plurality of cells may be modified, for example, by metabolic glycoengineering, to have site-specific functionalization of cell surfaces with azide groups, for example, without affecting cell viability. The DBCO on the surface of the microgel can thus react with an azide group as shown in FIG. 1B, where at least one second functional group on the microgel remains available and exposed for a reaction.

Each cell may be exposed to an engineered saccharide that comprises a first functional group, here shown as an azide group (N₃). In this example, the engineered saccharide is N-azidoacetylmannosamine-tetraacylated, which after the glycoengineering results in a surface-modified cell that bears the azide groups. After uptake of the modified saccharide comprising the first functional group, the cell metabolically processes the engineered saccharide. In this manner, the cell incorporates the saccharide comprising the first functional group into its cell membrane, where it may be exposed/presented at a surface of the cell and available for reaction.

As shown in the final process step, the dynamic cell support system may be formed by combining the microgel precursor beads bearing at least one second functional group (dibenzocyclooctyne (DBCO)) and the modified cells comprising at least one azide functional group, where they may be reacted to form bonds (e.g., covalent bonds) and thus annealed together to form a cellularized microgel (designated “cellular annealing”). Notably, the microgel precursor beads can also react with one another to form micro-annealed particles (MAP) defining a porous microgel that further incorporates the cells. In this manner, the present technology integrates glycoengineered cells as bioactive crosslinkers, allowing them to couple with microgels directly through bioorthogonal chemistry. This approach creates stable, biologically responsive structures that can be applied in tissue regeneration and engineered microtissues.

In certain variations, a dynamic cell support system may include a cellularized microgel. The dynamic cell support system may include a network comprising a microgel and at least one glycoengineered surface-modified cell attached to the microgel by a chemical (e.g., covalent) bond created between a first functional group on a surface of the glycoengineered surface-modified cell reacted with a second functional group on the microgel. While any of the chemistries identified above are contemplated, in certain aspects, the first functional group comprises an azide and the second functional group is an alkyne.

The present disclosure also contemplates a method for assembling microgels using metabolically glycoengineered cells as crosslinkers, forming structurally cohesive microtissues. Unlike traditional static crosslinking techniques, in certain variations, this method leverages strain-promoted azide-alkyne cycloaddition (SPAAC) chemistry, where azide-functionalized cells conjugate with DBCO-functionalized PEG microgels to form stable cell-gel networks. This system enhances the toolbox of micro-annealed particle (MAP) gel technology by enabling microgel-cell interactions through bio-orthogonal chemistry, allowing for controlled cell integration and stable network formation.

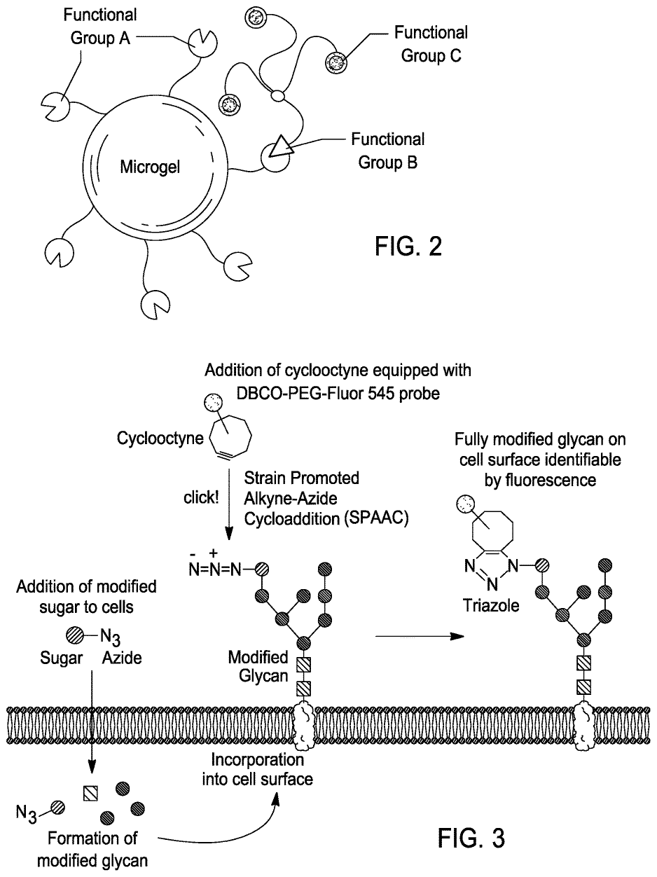

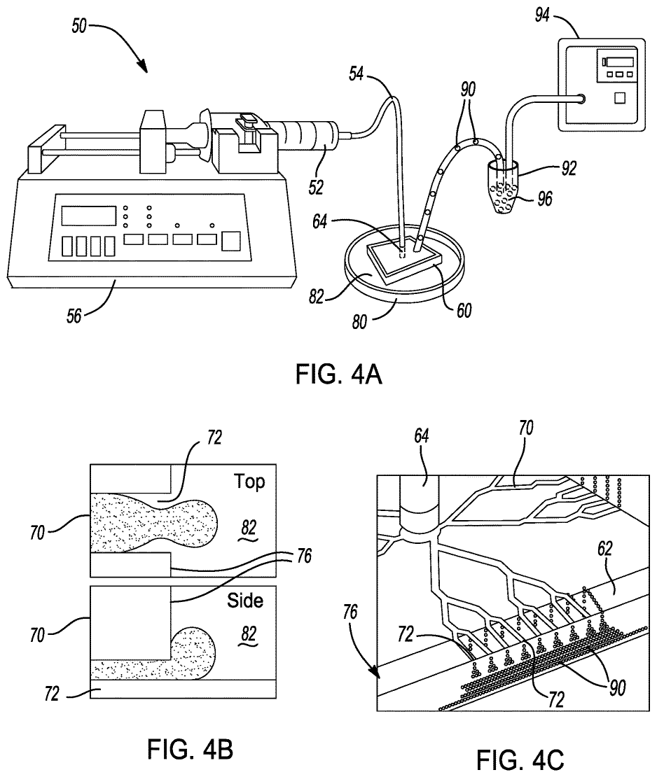

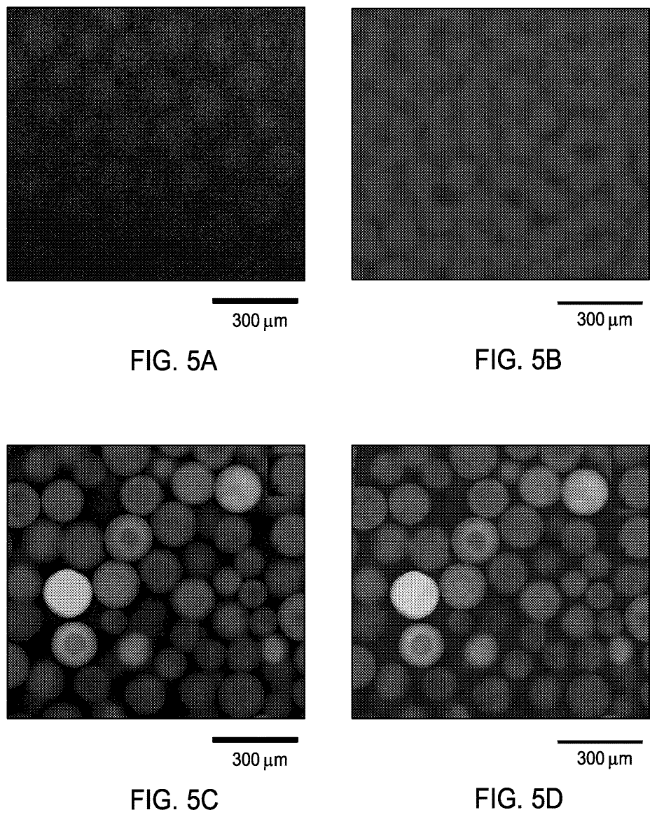

As described above, microgels, currently produced from (but not-limited to) polyethylene glycol (PEG)-based droplets, may be formed and crosslinked utilizing thiol-ene chemistry under 365 nm UV light. See for example, FIG. 2 that shows modification of a microgel precursor, similar to FIG. 1A, by reacting a microgel formed of PEG having functional group A (e.g., norbornene) or a PEG-Norbornene precursor and a PEG-dithiol precursor having functional group B (e.g., triazine) and functional group C (e.g., a dithiol), such that a thiol-ene click chemistry reaction may occur under UV light to form a PEG-microgel precursor where functional group A reacts with functional group B, leaving functional group C attached to the surface and available for reaction. The microgel further has at least one free functional group A (e.g., norbornene group) on the surface for further reaction according to certain aspects of the present disclosure. In certain aspects, DBCO modification of the microgel precursor (e.g., formed by a buoyancy-driven droplet generator shown in FIGS. 4A-4C) can include click chemistry under UV light that is employed to promote a reaction between PEG-Norbornene (microgel) and norbornene-tetrazine reaction through DBCO (3Arm)-PEG-Tetrazine (1Arm). Further, at least one free norbornene group remains on the surface of the microgel. As there are free norbornene groups on the surface of these microgels, DBCO functionalization via tetrazine-norbornene click chemistry is performed, which has been validated through fluorescence imaging (see FIGS. 5A-5D).

The annealable microparticles may be formed in a microfluidic system including a microfluidic device for forming a plurality of droplets, such as microdroplets, which may also be considered to be microparticles, that may be further processed as described above. For example, the microparticles may be subjected to a cross-linking or gelation reaction to become a microgel, as well as a reaction with a glycoengineered cell. The microfluidic system may include a microfluidic device for generating microdroplets for forming a microgel and is further described in co-pending and co-owned PCT Application No. PCT/US2026/23068 filed on Apr. 9, 2026 to Sasha Cai Lesher Perez, et al. entitled “BUOYANCY-CLEARED MICROFLUIDIC PLATFORM FOR RAPID AND UNIFORM DROPLET GENERATION AND MICROGELS FOR CELL AND TISSUE ENCAPSULATION,” the relevant portions of which are hereby incorporated by reference. For example, a microfluidic system 50 for generating microdroplets for a microgel includes a source of polymer or gel precursor 52 (as shown, the gel precursor is contained in a syringe as part of an automated syringe pump, although the syringe may be used manually to transfer the gel precursor, or other fluid transport systems may be used). The source of gel precursor 52 may feed a precursor stream 54. The microfluidic system 50 includes at least one pressure-generating component, which may be a pump, pneumatically-driven device or other automated pressure generation device or a manual-operation device, such as a manually operated syringe device. The pressure-generating component shown in FIG. 1A is an automated syringe pump 56. The syringe pump 56 pumps the precursor stream 54 so that it is introduced to a microfluidic fluid distribution or droplet generator component 60 that includes at least one layer 62. In certain aspects, the at least one layer is a first layer. In further variations, the droplet generator may have multiple layers in addition to the first layer. The precursor stream 54 is introduced into an inlet 64 (which may be centrally disposed) of the at least one layer 62 of the droplet generator component 60.

The inlet 64 may receive the precursor stream 54, which may be a fluid/liquid material prepared upstream (e.g., prior to being introduced into the source of gel precursor 52) and introduced to the inlet 64. In certain variations, the precursor stream 54 may comprise a liquid devoid of cells, while in other variations, the precursor stream 54 optionally further includes cells or tissue. The droplet generator component 60 also has a plurality of channels 70 in fluid communication with the inlet 64 and a plurality of outlets 72. In the variation shown, the outlets 72 are radially outward from the centrally disposed inlet 64. The outlets 72 may be disposed near a peripheral edge 76 of the droplet generator component 60. In this manner, the precursor material flows radially (outward) from the inlet 64 within the channels 70 of the at least one layer 62 of the droplet generator component 60. The channels 70 may have one or more branches formed therein, such that a first channel may be fluidly coupled to the inlet 64; however, they may be branched at least once and optionally multiple times (as shown, at least six times) and thus fluidly coupled to multiple outlets 72. In certain variations, the droplet generator component 60 may have greater than or equal to about 32 outlets, for example, optionally greater than or equal to about 32 outlets to less than or equal to about 2,048 outlets.

The channels 70 may be a microfluidic channel. A microfluidic channel 70 is formed in or on a substrate that has a cross-sectional area and volume sufficient to permit receiving, storing, and/or transferring materials, including fluids. Fluids include liquids, gases, vapors, and the like. Thus, a microchannel may have dimensions such that the length of the structure forms the greatest dimension, for example, a groove (an open shape) or channel (a structurally closed geometry). In certain variations, the microfluidic channels 70 may be fully enclosed structures defining a void region that permits fluid communication there through, as described further herein. The microfluidic channels 70 may have a variety of cross-sectional shapes, including circular, round, or oval (forming a tube or cylindrical shape), rectangular, including square, and the like.

The microfluidic system 60 further includes at least one fluid reservoir 80 for generating microdroplets for a microgel. More specifically, the microfluidic droplet generator component 60 may be at least partially disposed in the fluid reservoir 80, which may contain a fluid 82 that may be a static bath. In one variation, the fluid 82 may comprise 0.4 wt. % Picosurf® surfactant (a proprietary biocompatible surfactant commercially available from Sphere Fluidics) in Novec™ 7500 oil. At least a portion of the droplet generator component 60 contacts the fluid 82 bath. The fluid reservoir 80 of the microfluidic system 50 serves as the static bath and is configured to hold a fluid 82 having a distinct density from the precursor material. The second fluid in the reservoir 80 may be considered to be a continuous phase, while the first fluid in the precursor stream 54 may be considered to be a dispersed phase. As noted above, at least a portion of the droplet generator component 60 is disposed within or adjacent to the fluid reservoir 80, so that the plurality of outlets 72 are in fluid communication with the fluid 82 in the fluid reservoir 80. In this manner, the precursor stream/solution 54 exits the plurality of outlets 72 and enters the fluid 82 in the fluid reservoir 80 to form a plurality of microdroplets 90. As shown in FIGS. 4A-4C, the outlet 72 of the microchannel 70 facilitates droplet 90 formation, which in this variation, can float to an upper region of the fluid reservoir 80 while in the fluid 82 having a lower density and being more buoyant. The microdroplets 90 may be further collected from the reservoir 80. The microfluidic system 60 includes a collection vessel 92 that may be connected to a source of UV radiation 94 or other components for facilitating gelation or cross-linking reactions of a plurality of polyhedral microdroplets 93 to form a microgel 96 (e.g., a granular or porous hydrogel). The cells may also be added to a collection vessel 92 (or other container), where they may be subject to energy or heat from an external source to promote reaction and annealing.

By optimizing the metabolic glycoengineering of cells, a reproducible feeding strategy is established that ensures robust azide (functional group) incorporation, while maintaining cell viability. FIG. 3 shows a schematic of cell glycoengineering in accordance with certain aspects of the present disclosure, where a plurality of cells are exposed to a glycoprotein, here N-azidoacetylmannosamine-tetraacylated (Ac4ManNAz) at a concentration of about 50 micromoles (μM) in solution. Multiple feeding regimes were conducted from 1 to 3 days. More specifically, Ac4ManNAz sugars were dissolved in complete media as 50 micromoles (μM) solution, and this was fed to cells over multiple feeding regimes for 1-3 days as reflected in Table 2. A cyclooctyne equipped with DBCO-PEG-Fluor 545 probe is then introduced, which reacts with the azide group presented on the surface of the cell membrane. In this manner, after reaction, the modified glycan on the cell surface can be identified by fluorescence. Confocal and flow cytometry validated success of the uptake of the glycoprotein and incorporation into the cell membrane/surface. The cells were then taken for confocal imaging and flow cytometry with DBCO-PEG4-Fluor545 dye to validate the sugar incorporation and the sugar-shedding behavior over time.

TABLE 2

| | Day 0 | Day 1 | Day 2 | Evaluation |
|---|---|---|---|---|
| Condition | Feeding | Feeding | Feeding | Day |
| M₀M₁M₂F₃ | No | No | No | Day 3 |
| M₀M₁S₂F₃ | No | No | Yes | Day 3 |
| M₀S₁S₂F₃ | No | Yes | Yes | Day 3 |
| S₀S₁S₂F₃ | Yes | Yes | Yes | Day 3 |
| S₀S₁M₂F₃ | Yes | Yes | No | Day 3 |
| S₀M₁M₂F₃ | Yes | No | No | Day 3 |

Cell-gel coupling may be achieved by mixing azide-functionalized cells with DBCO-functionalized PEG microgels, as discussed above. Microgels can be interlinked together to create a long-lasting structure with inherent properties like porosity and injectability for wound healing applications. However, no microgel assembly techniques have integrated cells in a way that allows them to dictate microstructure formation and remodeling actively. Here, mechanically stable and cytocompatible networks are formed that allow cells to actively remodel the microstructure, facilitating the transition from a synthetic scaffold to a biologically integrated tissue-like construct. Such dynamic cell support systems may be used in various applications, including in tissue engineering and regenerative medicine.

Thus, the present technology has broad applications in regenerative medicine, where it can provide a platform for engineering microtissues with structural heterogeneity and biological functionality. Similarly, in drug delivery, microgel-cell networks can serve as carriers for localized, sustained release of therapeutic agents. Additionally, this system can be adapted for biosensing applications by integrating functionalized microgels with living cells to monitor biochemical changes in real-time. Moreover, the ability to form biologically integrated microstructures makes this approach advantageous for both in vitro studies and future in vivo applications.

In certain variations, the present disclosure contemplates a bio-orthogonal microgel assembly system, where glycoengineered cells act as active crosslinkers for PEG-based microgels. Azide-functionalized cells are generated via metabolic glycoengineering, while PEG microgels are synthesized using a buoyancy-driven microfluidic system and crosslinked via UV-initiated thiol-ene chemistry. Free norbornene groups on the microgels enable DBCO functionalization through tetrazine-norbornene click chemistry described above. Coupling both forms stable, bioactive networks, ensuring direct cell integration into microgel scaffolds. Thus, the present disclosure contemplates a cytocompatible, bio-orthogonal strategy for assembling microgels using glycoengineered cells as crosslinkers, enabling controlled cell integration and dynamic tissue-like organization.

In certain further aspects, the present disclosure relates to a method for making a dynamic cell support system. The method optionally includes reacting a first functional group on at least one glycoengineered surface-modified cell with a second functional group on a microgel or a precursor of the microgel to form a chemical bond between the glycoengineered surface-modified cell and the microgel to create the dynamic cell support system. As described above, the reacting further includes exposing the at least one glycoengineered surface-modified cell and the microgel or precursor of the microgel to an external source of energy (e.g., actinic radiation, e-beam) or heat to further promote the reaction.

In some aspects, the microgel or precursor of microgel includes a plurality of microparticles and the at least one glycoengineered surface-modified cell includes a plurality of at least one glycoengineered surface-modified cells and the reacting forms a cellularized microgel network. As noted above, the plurality of at least one glycoengineered surface-modified cells may serve as cross-linkers for the microgel particles in the network.

In one example, 50 microliters (μL) total volume of cell-microgel hybrid constructs formed with varying 10-250 million cells/mL of microgels through DBCO-Azide reaction are evaluated. More specifically, a 50 microliters (μL) cell-microgel hybrid mixture, prepared with varying cell densities (10-250 million cells/mL of microgels), was sandwiched between two polydimethylsiloxane (PDMS)-coated glass slides and incubated at 37° C. with 5% CO₂ for 3 hours to facilitate the DBCO-azide click reaction. After incubation, the slides were separated, and the assembled structures were subjected to vigorous PBS flushing to test the stability of the DBCO-azide-mediated assemblies.

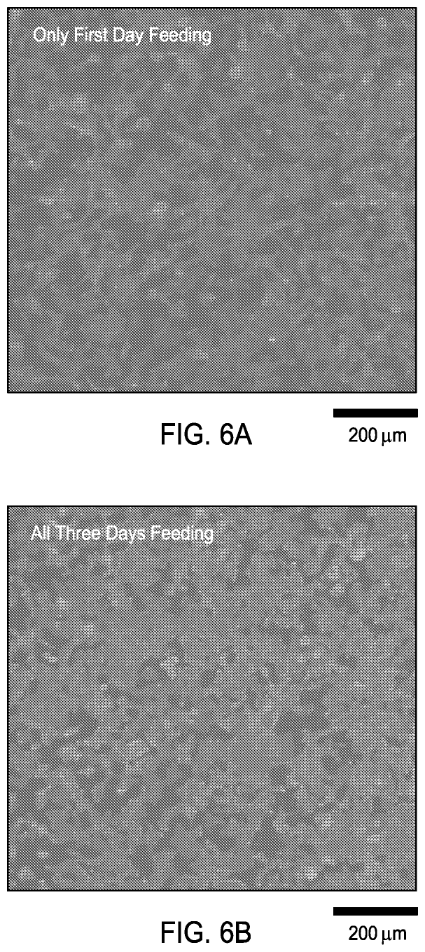

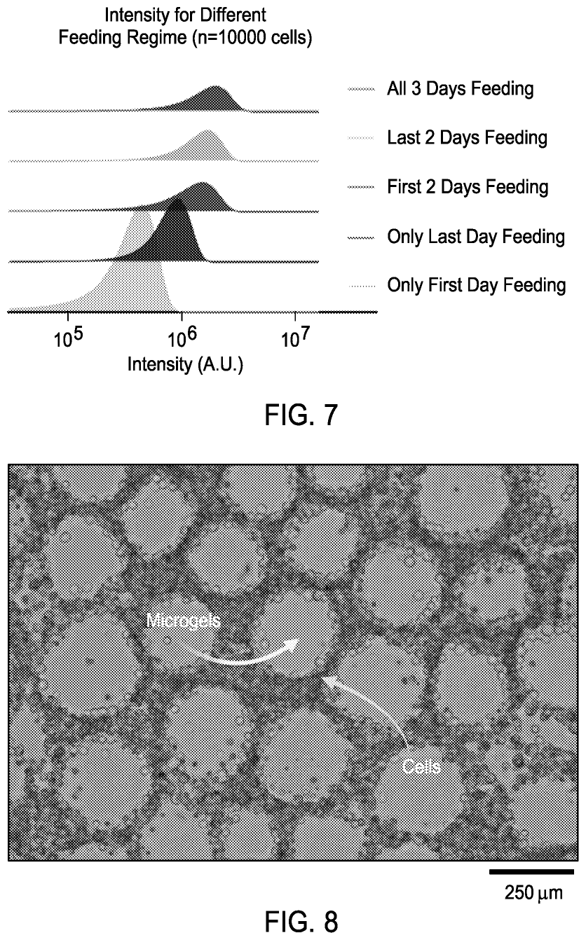

A time sweep rheology is done on 8 mm parallel plate under 5% strain for 3 hours. DBCO modified beads are stained with AzideFluor 488 and visualized under confocal microscopy. FIG. 6 shows signals are localized across membrane surfaces, indicating robust glycoengineering. The cells described above were taken for confocal imaging and flow cytometry with DBCO-PEG4-Fluor545 dye to validate the sugar incorporation and the sugar-shedding behavior over time. FIG. 6 compares intensity of images for cells after a first day of feeding and after three days of feeding with a glycoprotein, where the three days results in greater intensity. FIG. 7 shows a progressive shift in fluorescence intensity from Day 1 to Day 3. The highest azide expression was observed with 3 consecutive days of Ac4ManNAz feeding. The results validate that surface azide density is feeding-dependent, enabling tunable control of cell-microgel coupling efficiency. FIG. 8 shows successful formation of a dynamic cell support system include azide-functionalized cells incorporated into a microgel matrix. More specifically, FIG. 8 shows coupling of DBCO-modified microgels with azide sugar-fed glycoengineered cells using strain-promoted azide-alkyne cycloaddition (SPAAC) click chemistry. As such, azide-functionalized cells can serve as living, dynamic crosslinkers for microgels. Further, the dynamic cell support system of the present disclosure maintains structure and supports the creation of modular, cell-integrated microtissues.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A dynamic cell support system comprising:
at least one modified cell having a modified surface comprising a first functional group; and
a microgel comprising a second functional group capable of reacting with the first functional group to incorporate the at least one modified cell into the microgel and create the dynamic cell support system.

2. The dynamic cell support system of claim 1, wherein the at least one modified cell has been modified by uptake of a saccharide comprising the first functional group, wherein the at least one modified cell incorporates the saccharide comprising the first functional group into its cell membrane.

3. The dynamic cell support system of claim 1, wherein the first functional group comprises an azide and the modified surface is an azide-modified surface.

4. The dynamic cell support system of claim 1, wherein the first functional group comprises tetrazine.

5. The dynamic cell support system of claim 1, wherein the first functional group is an azide and the second functional group is an alkyne.

6. The dynamic cell support system of claim 1, wherein the second functional group comprises dibenzocyclooctyne or norbornene.

7. The dynamic cell support system of claim 1, wherein the microgel comprises a polymer selected from the group consisting of: hyaluronic acid, polyalkylene glycol, alginate, gelatin, trehalose, and combinations thereof, the polymer further has a functional group or moiety selected from the group consisting of: norbornene, maleimide, cyclononane, bicyclononyne, dibenzocyclooctyne (DBCO), dibenzoazacyclooctyne (DIBAC), vinyl sulfone, azide, tetrazine, acrylate, methacrylate, and combinations thereof.

8. The dynamic cell support system of claim 1, wherein the microgel comprises a polymer selected from the group consisting of: polyalkylene glycol, hyaluronic acid, and combinations thereof.

9. The dynamic cell support system of claim 1, wherein the microgel comprises a polymeric precursor selected from the group consisting of: hyaluronic acid-norbornene, polyethylene glycol-norbornene, polyethylene glycol-bicyclononyne, polyethylene glycol-tetrabicyclononyne, polyethylene glycol-maleimide, polyethylene glycol-di-acrylate, polyethylene glycol-tetra-acrylate, gelatin-norbornene, gelatin-methacrylate, alginate, trehalose, and combinations thereof.

10. A dynamic cell support system comprising:
a porous network comprising a microgel and at least one glycoengineered surface-modified cell attached to the microgel by a chemical bond created between a first functional group on a surface of the glycoengineered surface-modified cell reacted with a second functional group on the microgel.

11. The dynamic cell support system of claim 10, wherein the first functional group comprises an azide and the second functional group is an alkyne.

12. The dynamic cell support system of claim 10, wherein the first functional group comprises an azide and the second functional group comprises dibenzocyclooctyne (DBCO).

13. The dynamic cell support system of claim 10, wherein the at least one glycoengineered surface-modified cell is a plurality glycoengineered surface-modified cells and at least a portion of the plurality glycoengineered surface-modified cells serve as cross-linkers between polymer chains in the microgel.

14. The dynamic cell support system of claim 10, wherein the first functional group is an azide and the second functional group is an alkyne.

15. The dynamic cell support system of claim 10, wherein the second functional group comprises dibenzocyclooctyne or norbornene.

16. The dynamic cell support system of claim 10, wherein the microgel comprises a polymer selected from the group consisting of: hyaluronic acid, polyalkylene glycol, alginate, gelatin, trehalose, and combinations thereof, the polymer further has a functional group or moiety selected from the group consisting of: norbornene, maleimide, cyclononane, bicyclononyne, dibenzocyclooctyne (DBCO), dibenzoazacyclooctyne (DIBAC), vinyl sulfone, azide, tetrazine, acrylate, methacrylate, and combinations thereof.

17. The dynamic cell support system of claim 10, wherein the microgel comprises a polymeric precursor selected from the group consisting of: hyaluronic acid-norbornene, polyethylene glycol-norbornene, polyethylene glycol-bicyclononyne, polyethylene glycol-tetrabicyclononyne, polyethylene glycol-maleimide, polyethylene glycol-di-acrylate, polyethylene glycol-tetra-acrylate, gelatin-norbornene, gelatin-methacrylate, alginate, trehalose, and combinations thereof.

18. A method for making a dynamic cell support system, the method comprising:
reacting a first functional group on at least one glycoengineered surface-modified cell with a second functional group on a microgel or a precursor of the microgel to form a chemical bond between the glycoengineered surface-modified cell and the microgel to create the dynamic cell support system.

19. The method of claim 18, wherein the reacting further comprises exposing the at least one glycoengineered surface-modified cell and the microgel or precursor of the microgel to an external source of energy or heat.

20. The method of claim 18, wherein the microgel or precursor of microgel comprises a plurality of microparticles and the at least one glycoengineered surface-modified cell comprises a plurality of at least one glycoengineered surface-modified cells and the reacting forms a cellularized microgel network.
